(19) United States
(12) Patent Application Publication
(10) Pub. No.: US 2021/0302427 A1
(43) Pub. Date: Sep. 30, 2021

(54) Secure Immunity Information Transmission System And Network

(71) Applicant: Shuhari Group, LLC, Nevis (KN)

(72) Inventor: Lucas Stone, Cheyenne, WY (US)

(73) Assignee: Shuhari Group, LLC, Nevis (KN)

(21) Appl. No.: 17/218,088

(22) Filed: Mar. 30, 2021

Related U.S. Application Data
(60) Provisional application No. 63/002,979, filed on Mar. 31, 2020.

(51) Int. Cl. G01N 33/569

(52) U.S. Cl. CPC G01N 33/56983; G01N 2333/165

(57) ABSTRACT

A method for managing an immune pass includes receiving uploaded immunization data for at least one individual, wherein the immunization data comprises data indicating antibody levels for a particular virus. The method also includes analyzing the immunization data to determine an immune pass status for the at least one individual and generating an immune pass for the at least one individual, wherein the immune pass includes the immune pass status and personal identifiable information for the at least one individual. The method further includes storing the immune pass, verifying a request to retrieve the immune pass corresponding to the at least one individual, and transmitting the immune pass in response to verification of the request.

### CROSS-REFERENCE TO RELATED APPLICATIONS

The present application claims the benefit of U.S. Provisional Patent Application No. 63/002,979, entitled “SECURE IMMUNITY INFORMATION TRANSMISSION SYSTEM AND NETWORK” and filed Mar. 31, 2020, the entire disclosure of which is incorporated herein by reference for all purposes.

### BACKGROUND

With the past, current and potential future onslaught of global infectious disease so acutely displayed in the present by SARS-CoV-2 aka COVID-19, as well as SARS-CoV-1, MERS, H1N1, and HIV, among others, in recent decades, a large portion of individuals cannot or will not venture out of their homes into private and/or public spaces to reduce a risk of contracting or spreading the SARS-CoV-2 virus. Due to the ease of transmission of SARS-CoV-2, individuals without antibodies to the virus are at high risk of contracting the virus when in proximity with another individual carrying the virus. As some individuals are asymptomatic or show only minor symptoms to COVID-19, it is very difficult for individuals to gather in private and/or public spaces safely. Exposure and survival to COVID-19 may generate antibodies to the disease. Some tests may be able to detect measurable levels of antibodies in an individual's blood. If these antibodies are present in sufficient quantity, then infectious transmission in the organism is greatly minimized. Groups of individuals and/or animals with similar levels of antibodies may be able safely congregate with minimized risk of disease transmission or disease re-occurrence. However, the private and/or public spaces may generally have individuals with antibodies and without antibodies making these spaces potentially hazardous to individuals.

### BRIEF DESCRIPTION OF THE DRAWINGS

These drawings illustrate certain aspects of some of the embodiments of the present invention and should not be used to limit or define the invention.

FIG. 1 illustrates a block diagram of the immune pass system, in accordance with embodiments of the present disclosure.

FIG. 2 illustrates a flow chart for managing the immune pass system, in accordance with embodiments of the present disclosure.

FIG. 3 illustrates a flow chart for requesting an immune pass using the immune pass system, in accordance with embodiments of the present disclosure.

FIG. 4 illustrates a front view of the cellular device showing a map overlay feature of the mobile application, in accordance with embodiments of the present disclosure.

FIG. 5 illustrates a front view of a cellular device displaying a request tool for a mobile application of the immune pass system, in accordance with embodiments of the present disclosure.

FIGS. 6A-6C illustrate front views of respective immune pass formats displayed on a mobile device via the mobile application, in accordance with embodiments of the present disclosure.

### DETAILED DESCRIPTION

It is to be understood that the present disclosure is not limited to particular systems or methods, which may, of course, vary. It is also to be understood that the terminology used herein is for the purpose of describing particular embodiments only and is not intended to be limiting. All numbers and ranges disclosed herein may vary by some amount. Whenever a numerical range with a lower limit and an upper limit is disclosed, any number and any included range falling within the range are specifically disclosed. Although individual embodiments are discussed herein, the invention covers all combinations of all those embodiments. As used herein, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected. If there is any conflict in the usages of a word or term in this specification and one or more patent or other documents that may be incorporated herein by reference, the definitions that are consistent with this specification should be adopted for the purposes of understanding this invention.

Disclosed herein are immune pass systems and methods for managing (e.g., uploading, storing, requesting, transmitting, and providing) antibody data for at least one virus (e.g., SARS-CoV-2, SARS-CoV-1, MERS, H1N1, or HIV) via a global or regional network. The network is configured to digitally store immune passes (e.g., digital certificates) having immunization data (e.g., antibody data, vaccination data, personal identifiable information) for individuals. In particular, the network is configured to digitally store immune passes for individuals having sufficient immunity to a particular virus (e.g., one or more strains of the virus). Upon request, a corresponding immune pass may be digitally transmitted to an individual for use in accessing an immunity restricted space (e.g., workplace, schools, universities, hospitals, clinics, daycare, houses of worship, athletic facilities, entertainment venues, hotels, temporary housing, airplanes, cruise ships, public venues, house party, public transportation, farms, temporary pet housing, dating website application, online community, etc.). Providing the immune pass may permit access to the immunity restricted space based on criteria (e.g., antibody levels, B-cell levels, T-cell levels, vaccination status, time since antibody test, B-cell test, T-cell test, or vaccination, etc.) set by the immunity restricted space.

The immune pass systems and methods may allow for individuals to gather in private and/or public spaces more safely during the current pandemic. For various reasons, the current pandemic has had a devastating effect on many businesses. One reason is that the number of customers visiting these businesses has sharply decreased. A large portion of individuals cannot or will not venture out into private and/or public spaces to reduce a risk of contracting or spreading the SARS-CoV-2 virus. However, individuals having sufficient antibodies, B-cells, and/or T-cells for the SARS-CoV-2 virus could potentially venture out into private and/or public spaces to visit these businesses without substantial risk of contracting, and possibly spreading, the SARS-CoV-2 virus. The present systems and methods may help identify and provide access to businesses having immunity restricted spaces, accessible via the immune pass, where these individuals may more safely visit and support these businesses.

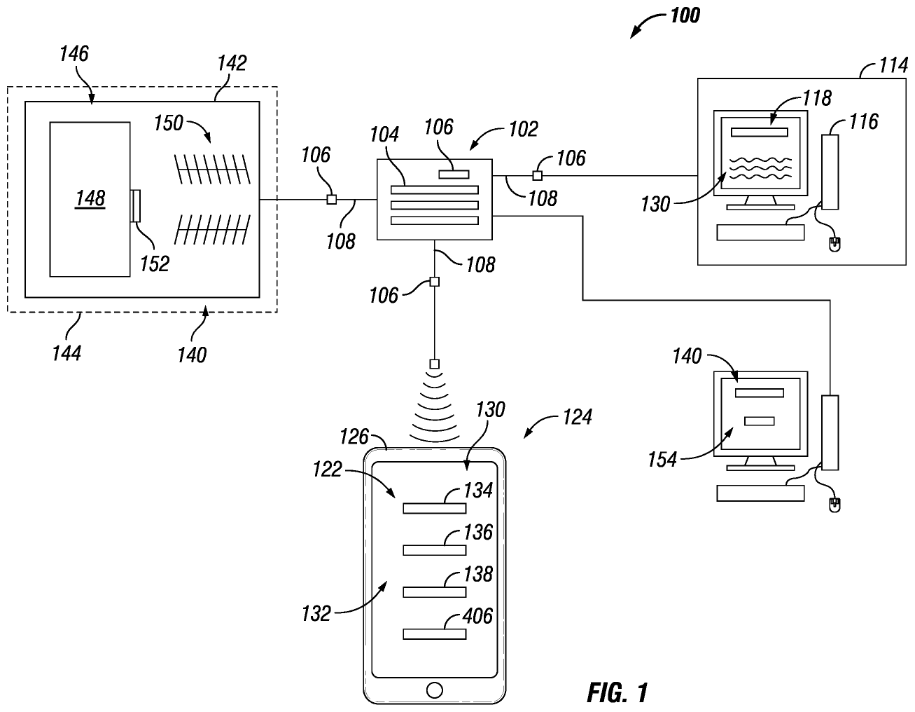

FIG. 1 illustrates a block diagram of the immune pass system 100, in accordance with embodiments of the present disclosure. As illustrated, the immune pass system 100 (e.g., a secure immunity information transmission system and network) includes a secure data storage system 102 accessible via the internet. The secure data storage system 102 may include any suitable data storage system 102 (e.g., a cloud storage system, a data storage center, etc.). Further, the secure data storage system 102 may be configured to store data compliant with HIPAA or other medical/veterinary rules and regulations guided by the USFDA and other global regulatory bodies, based at least in part on a location of usage of the immune pass system 100. The secure data storage system 102 may include servers 102, routers 104, security software, firewalls, computing systems 106, communication devices 108 (e.g., wired connections, wireless connections, etc.), and/or any suitable components for storing and securing data in the secure data storage system 102.

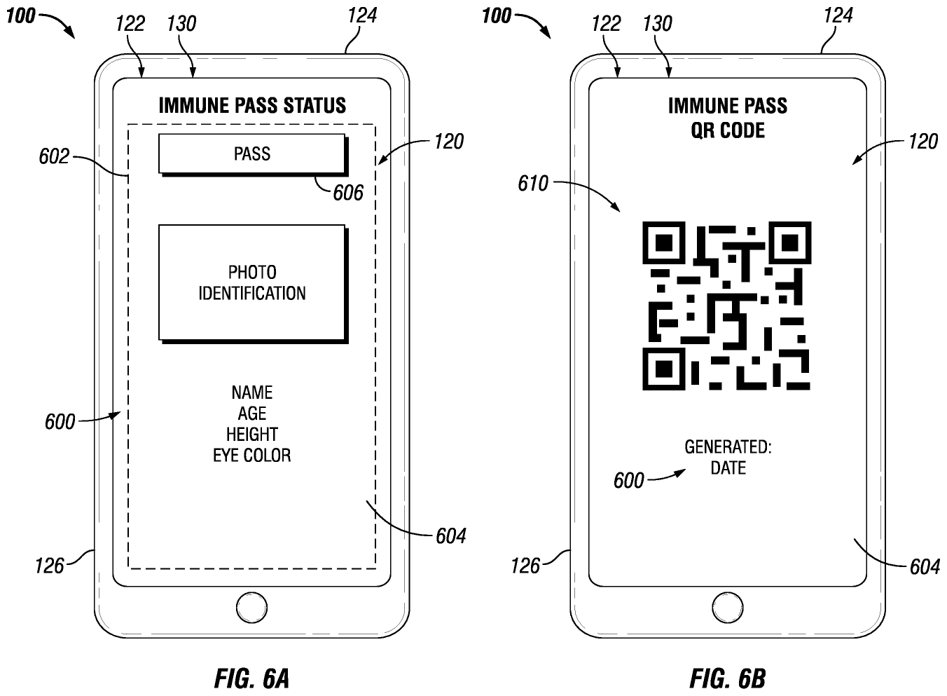

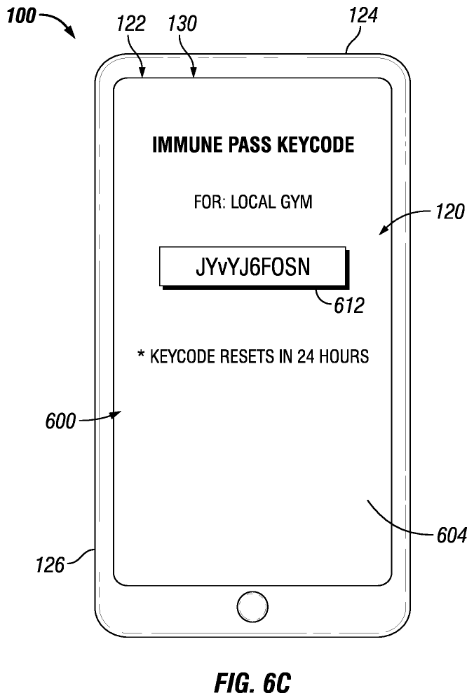

The secure data storage system 102 may be configured to store immunization data (e.g., antibody data, B-cell data, T-cell data, vaccination data, personal identifiable information) for individuals. For example, the secure data storage system 102 may be configured to store antibody titer test results (e.g., antibody data) and/or other test results for the individuals. Further, the secure data storage system 102 may be configured to store personal identifiable information and/or contact information for individuals corresponding to the antibody data, B-cell data, T-cell data, and/or vaccination data. The personal identifiable information may include photo identification, name, birth date, social security number, license number, etc. At least some of the personal identifiable information may be included in an immune pass 120 (shown in FIGS. 6A-6C) for identification and/or verification purposes at the immunity restricted space 140. Moreover, the contact information may include address, phone number, email address, etc. for the individual. In some embodiments, the contact information may be used for account recovery, antibody test verification, B-cell test verification, T-cell test verification, vaccination verification, or other purposes.

As illustrated, the immune pass system 100 may include devices 124 configured to connect to the secure data storage system 102. In some embodiments, a health care provider 114 may have a computing device 116 configured to connect to the secure data storage system 102 via the internet. In some embodiments, the health care provider 114 may upload immunization data to the secure data storage system 102 via the computing device 116. The computing device 116 may be configured to upload the immunization data via the secure data storage system 102 via an immune pass account accessible via a web application 118 or another suitable interface of the immune pass system 100. As set forth above, the immunization data may include antibody data, B-cell data, T-cell data, and/or vaccination data for an individual to a particular virus. For example, the immunization data may include titer test results with antibody levels for the SARS-CoV-2 virus. The secure data storage system 102 may be configured to determine an immunity status and/or immunity rating for the individual based at least in part on the immunization data. Using the immunization data, the personal identifiable information, and/or the contact information for the individual, the secure data storage system 102 may be configured to generate the immune pass 120.

The immune pass 120 may be retrievable by the individual corresponding to the immune pass 120. However, in some embodiments, the immune pass 120 may be retrievable via a third party having written permission from the individual. The immune pass system 100 may require verification of the written permission via confirmation sent directly to the individual via the stored contact information and/or a notification sent to an immune pass account associated with the individual.

Individuals and health care providers 114 may register for an immune pass account via a mobile application 122, the web application 118, or another suitable interface associated with the immune pass system 100. For an individual, the immune pass account associated with the individual may be linked to a corresponding immune pass 120, as well as other information associated with the individual. As set forth above, the immune pass 120 may be retrievable by the individual corresponding to the immune pass 120. In some embodiments, the individual may access their immune pass account to retrieve their immune pass 120 via a device 124 (e.g., mobile phone 126, tablet, computer, smart watch, etc.). The application 130 (e.g., mobile application 122, web application 118, etc.) of the immune pass system 100 may be installed or accessible via the device 124.

In the illustrated embodiment, the immune pass system 100 includes a mobile device 124 configured to access the immune pass account of the individual, which may include a tool for requesting the immune pass 120 associated with the individual. The mobile application 122 may include a user interface (UI) configured to display the mobile application 122 on a mobile device 124. Upon loading the mobile application 122, the user interface may display a login screen of a login system. The login system may log into the immune pass account corresponding to the individual in response to input of corresponding credentials (e.g., username, email, password, etc.) for the immune pass account. After successfully logging into the immune pass account, the mobile application 122 may be configured to display a main menu 132 having UI buttons for various tools of the mobile application 122. For example, the mobile application 122 may have a first UI button 134 for an immunity test upload tool configured to upload immunization data for the individual (e.g., antibody data, B-cell data, T-cell data, vaccination data). Further, the mobile application 122 may include a second UI button for a request tool. The request tool may be configured to output a request to retrieve the immune pass 120 corresponding to the immune pass account logged into the mobile application 122. Additionally, the mobile application 122 may include a third UI button 138 for an output feature configured to output (e.g., display, email, message, etc.) the immune pass 120.

In some embodiments, the mobile application 122 may be configured to output the immune pass 120 via displaying the immune pass 120. For example, the mobile application 122 may show an image of the immunity status and/or immunity rating for the individual, as well as personal identifiable data for the individual, which may be used to access an immunity restricted space 140. Alternatively, the mobile application 122 may be configured to show a QR code having the immunity status and/or immunity rating for the individual, as well as personal identifiable data for the individual, that may be used to access the immunity restricted space 140. In another embodiment, the mobile application 122 may be configured to output a keycode that may be used to access the immunity restricted space 140. For example, the keycode may be configured to provide access to an immunity restricted dating website. In some embodiments, the mobile application 122 may include an option for the individual (e.g., user) to select a type of output for the immune pass 120, as a required output for the immune pass 120 may vary based on the immunity restricted space 140. In some embodiments, mobile application may be configured to order an immune pass (e.g., an RFID tag) configured to transmit a signal indicating the immune pass status of the individual. In some embodiments, the immune pass may be used with animals (e.g., cows, horses, etc.) to identify animals that have sufficient immunity status and/or immunity rating.

The immunity restricted space 140 may include a physical space 142 having a corresponding geofenced area 144. For example, the immunity restricted space 140 may include a workout facility 146. The building 148 and/or parking lot 150 corresponding to the workout facility 146 may form the geofenced area 144. The workout facility 146 may include a requestor 152 (e.g., scanner, reader, receiver, etc.) configured to scan the immune pass 120. For example, the workout facility 146 may have a QR code scanner configured to read a QR code (e.g., having the immune pass 120) displayed on the mobile device 124 of the individual entering the workout facility 146. Based at least in part on the scan of the immune pass 120, the workout facility 146 may permit entrance of the individual to the workout facility 146. In some embodiments, the immunity restricted space 140 may include a virtual space 154 (e.g., dating applications, online communities, job applications, online registration, etc.).

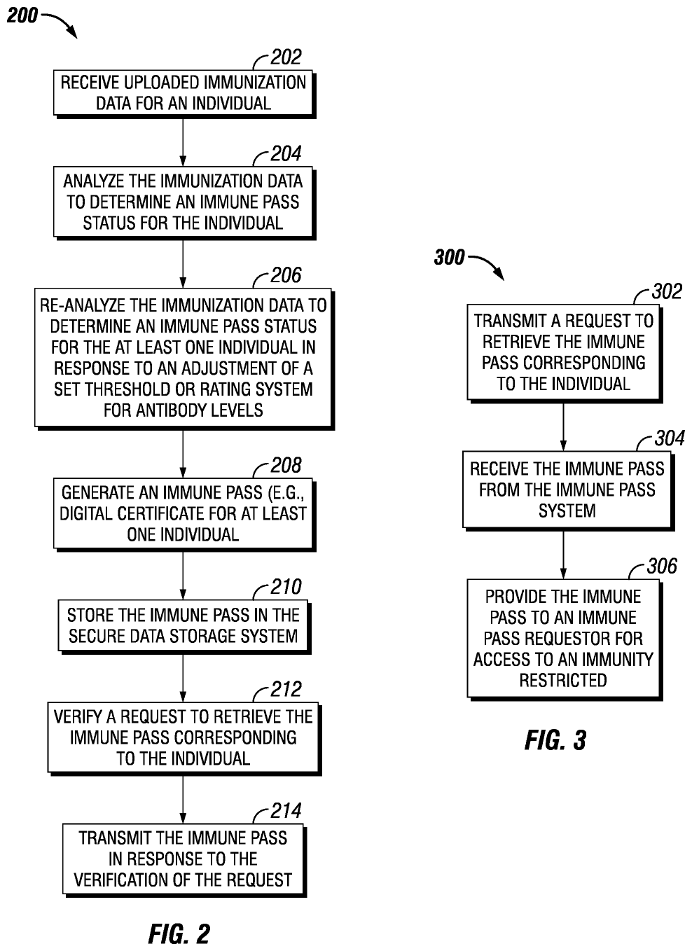

FIG. 2 illustrates a flow chart of a method 200 for managing the immune pass system, in accordance with embodiments of the present disclosure. The method includes the step 202 of receiving uploaded immunization data for an individual. That is, the immune pass system may be configured to receive immunization data uploaded by a health care provider via the immune pass account corresponding to the health care provider. The immunization data may include vaccination data (e.g., date of vaccination, vaccine information, etc.) and/or antibody data (e.g., data indicating antibody levels for a particular virus obtained via a titer test), B-cell data, and/or T-cell data for a particular virus (e.g., the SARS-CoV-2 virus).

The method may further include the step 204 of analyzing the immunization data to determine the immunity status (e.g., immune pass status) for the individual. In some embodiments, the immune pass status may include a pass status or fail status. The pass status may indicate that the individual has antibody levels, B-cell levels, and/or T-cell levels, at or above a set threshold and/or that the individual has received a corresponding vaccine. The fail status may indicate that the individual has an antibody, B-cell, and/or T-cell levels below the set threshold and/or the vaccine has not been received or has expired. The set threshold may be a uniform threshold value set by the immune pass system. However, in some embodiments, the set threshold may vary throughout the immune pass system. In some embodiments, the set threshold may vary based at least in part on a location of the individual. For example, each immunity restricted space may submit a desired antibody threshold level or vaccination window to the immune pass system via the immune pass account corresponding to the immunity restricted space. The immune pass system may be configured to compare the antibody level of the individual against the desired antibody threshold level for the immunity restricted space corresponding to the location of the individual. In some embodiments, the immune pass system may be configured to compare the date the vaccination was received by the individual against the vaccination window corresponding to the location of the individual.

Alternatively, the immune pass status may include the immunity rating. The immunity rating may be determined based at least in part on a rating system for antibody levels, B-cell levels, T-cell levels, or some combination thereof. In one example, the immunity rating may be based solely on antibody levels with the rating system assigning a first rating to a first range of antibody levels, a second rating to a second range of antibody levels, and a third rating to a third range of antibody levels. The second range may include higher antibody levels than the first range, and the third range may include higher antibody levels than the second range. In some embodiments, immunity restricted spaces may require at least a specific rating for permitting entrance. For example, a first immunity restricted space may require at least a second rating for entrance and a second immunity restricted space may require at least a third rating for entrance. In some embodiments, the method further includes the step 206 of re-analyzing the immunization data to determine an immune pass status for the at least one individual in response to an adjustment of a set threshold or rating system for antibody levels.

Moreover, the method further includes the step 208 of generating an immune pass (e.g., digital certificate) for the at least one individual. The immune pass may include the immune pass status and personal identifiable information for the at least one individual. As set forth above, the immune pass status may include a pass/fail indication, the immunity rating, or some combination thereof. In some embodiments, the immune pass system may be configured to generate the immune pass in response to determining the immune pass status for the individual. Alternatively, the immune pass system may generate the immune pass in response to a request to retrieve the immune pass. As set forth above, the immunity restricted spaces may require the immune pass to be provided through various formats/mediums (e.g., QR code, keycode, text and/or image-based display, etc.). Further, each of the immunity restricted spaces may require varying amount of personal identifiable information and/or contact information. For example, a first immunity restricted space may require the immune pass to provide only a name of the individual with the immune pass status. However, a second immunity restricted space may require the immune pass to provide a name, age, height, eye color, as well as the immune pass status. In some embodiments, the request to retrieve the immune pass may include criteria for the immune pass. In response to receiving the request (e.g., having set criteria) to retrieve the immune pass, the immune pass system may be configured to generate the immune pass based at least in part on the criteria for the immunity restricted space. The criteria for the immune pass may include the type of information required as well as the format/medium for providing the immune pass. Moreover, the immune pass system may be configured to generate a default immune pass for immunity restricted spaces with unspecified criteria. Once the immune pass is generated, the method further includes the step 210 of storing/saving the immune pass in the secure data storage system.

The method further includes the step 212 of verifying a request to retrieve the immune pass corresponding to the individual. The immune pass system may include software and/or firewalls configured to protect information (e.g., personal identifiable information, contact information, immune pass status, etc.) stored in the secure data storage system. In some embodiments, the mobile application or web application making the request may send verification data based at least in part on the immune pass account logged into the application. In some embodiments, the immune pass system may require further verification data to pass on the request to the secure data storage system. The immune pass system may require biometric data of the individual operating the application to make the request. For example, the individual may log into their corresponding immune pass account via the mobile application on a mobile device. The individual may initiate a request to retrieve their corresponding immune pass. In response to initiating the request, the mobile application may output a prompt instructing the individual to perform a biometric scan (e.g., facial recognition, thumbprint scan, etc.). The mobile application may be configured to send biometric data associated with the biometric scan as verification data for the request to retrieve the immune pass.

The method further includes the step 214 of transmitting the immune pass in response to verification of the request. The immune pass system may be configured to transmit the immune pass to the device making the request via the mobile application or web application. That is, the immune pass may be accessible on the device via the mobile application or web application loaded on the device. Further, the immune pass may only be accessible on the device via the application when the corresponding immune pass account is logged into the device.

FIG. 3 illustrates a flow chart of a method 300 for requesting an immune pass using the of the immune pass system, in accordance with embodiments of the present disclosure. The method includes the step 302 of transmitting a request to retrieve the immune pass corresponding to the individual. The individual may make the request via a mobile application or web application loaded on a suitable device (e.g., mobile phone, computer, tablet, etc.). For example, the individual may load the web application on their mobile phone and login to their corresponding immune pass account. The individual may select a UI option to output request to retrieve the immune pass data. As set forth above, the mobile application may prompt the individual to perform a biometric scan before sending the request. In some embodiments, the mobile application may prompt the individual to provide a pin code or another layer of verification before sending the request.

As set forth above, the immune pass system may be configured too output the immune pass to the mobile application in response to the request. Accordingly, the method further includes the step 304 of receiving the immune pass from the immune pass system. In particular, the step may include receiving the immune pass from the secure data storage system of the immune pass system. After receiving the immune pass, the immune pass may be accessible via the mobile application on the device. That is, the individual may select another UI option in the mobile application to display the immune pass.

The method further includes the step 306 of providing the immune pass to an immune pass requestor for access to an immunity restricted space. For example, the immunity restricted space may include a public library. The public library may have a requestor (e.g., scanner) disposed at the entrance of the library. The individual may provide the immune pass displayed on the screen of the mobile device, via the web application, for access to the public library. The scanner may read the immune pass and determine that the individual has an acceptable immune pass status to enter the public library.

In some embodiments, the immunity restricted space includes a physical space having a corresponding geofenced area. The mobile application may be configured to provide the immune pass in response to entering the geofenced area. For example, the mobile application, via the device, may detect that the device associated with the individual has entered the geofenced area and automatically provide the immune pass corresponding to the immune pass account logged into the mobile application. The mobile application may be configured to output a wireless signal having the immune pass to the requestor associated with the geofenced area. Alternatively, or additionally, the mobile application may be configured to output the request to retrieve the immune pass associated with the immune pass account in response to entering the geofenced area. The mobile application may be configured to receive criteria for the immunity restricted space to include in the request in response to entering the immunity restricted space.

Moreover, in some embodiments, the immunity restricted space includes a virtual space (e.g., dating applications, online communities, job applications, online registration, etc.). The mobile application may be configured to provide a keycode corresponding to the immune pass for the individual to enter in a mobile application or website corresponding to the virtual space for admittance into the virtual space.

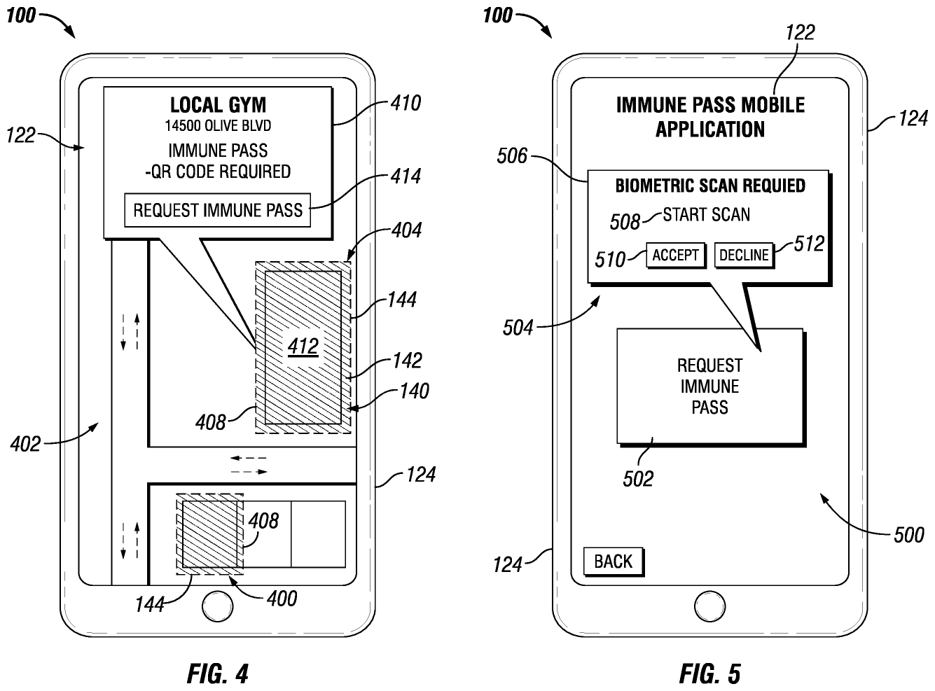

FIG. 4 illustrates a front view of the cellular device 124 showing a map overlay feature 400 of the mobile application 122, in accordance with embodiments of the present disclosure. As set forth above, the immunity restricted space 140 may include a physical space 142 having a corresponding geofenced area 144. The mobile application 122 may be configured to request and/or provide the immune pass 120 to the requestor 152 (e.g., shown in FIG. 1), associated with the immunity restricted space 140 corresponding to the geofenced area 144, in response to entering the geofenced area 144. Further, the mobile application 122 may be configured to receive data (e.g., required criteria, etc.) from the immunity restricted space 140 in response to entering the geofenced area 144.

As illustrated, the mobile application 122 may include the map overlay feature 400 configured to overlay the geofenced area 144 onto a digital map 402. For example, the mobile application 122 may have a fourth UI button 406 on the main menu 132 of the mobile application 122 configured to open the map overlay feature 400. The map overlay feature 400 may show a street map or other suitable digital map 402. The map overlay feature 400 may further include geofenced areas 144 displayed on the digital map 402. As illustrated, the geofenced areas 144 may include a dashed border 408 surrounding each geofenced area 144. The dashed border 404 may include a tooltip 410 for the geofenced area 144. The tooltip 410 is configured to provide information regarding the immunity restricted space 140 corresponding to the geofenced area 144. For example, the individual may select a region 412 on the map overlay feature 400 within a first geofenced area 402. In response to the selection, the map overlay feature 400 may display the tooltip 410 showing the name of the immunity restricted space 140 (e.g., generic gym), the address, and immune pass requirements [e.g., time since last request, level of immunity, acceptable immune pass mediums (digital image via phone, QR code, printed, RFID), etc.] for the immunity restricted space 140. In some embodiments, the tooltip 410 may include a fifth UI button 414 corresponding to an option to request an immune pass 120 for the immunity restricted space 140 corresponding to the selection. Pre-requesting the immune pass 120 may store the immune pass 120 that meets the criteria for the immunity restricted zone 140, such that the individual may provide the immune pass 120 to immunity restricted spaces 140 that may be disposed outside of cell phone data coverage.

FIG. 5 illustrates a front view of a cellular device 124 displaying a request tool 500 for a mobile application 122 of the immune pass system 100, in accordance with embodiments of the present disclosure. As set forth above, the request tool 500 of the application (e.g., mobile application 122 or web application 118) of the immune pass system 100 may be configured to output the request to retrieve the immune pass 120 corresponding to the individual associated with the account logged into the application. The request tool 500 may be accessible via selecting the second UI button 136 associated with the request tool 500 from the main menu 132 of the application 130 (e.g., shown in FIG. 1). Alternatively, second UI button 136 may be configured to output the request as set forth below.

As illustrated, the request tool 500 may include a sixth UI button 502 configured to submit the request to retrieve the immune pass 120 in response to user input. The request tool 500 may also include a biometric scan feature 504. In some embodiments, the request tool 500 may be configured to output a prompt 506 to perform a biometric scan (e.g., facial recognition, thumbprint scan, etc.) in response to selecting the sixth UI button 502 to submit the request. The immune pass system 100 may generally require biometric data to verify the request. However, in some embodiments, the immune pass system 100 may selectively require biometric data based at least in part on criteria set by an immunity restricted space 140. The request tool 500 may be configured to output the prompt 506 for the biometric scan in response to determining that the criteria for the immunity restricted space 140 includes biometric verification.

The prompt 506 to perform the biometric scan may include an indication 508 configured to inform the individual that a biometric scan is required to send the request. The prompt 506 may further include a UI accept button 510 corresponding to an option to proceed with performing the scan. In response to selecting the UI accept button 510, the application may be configured to launch a biometric scanner (e.g., facial recognition, thumbprint scanner, etc.) associated with the device 124. The mobile application 122 may be configured to upload biometric data from the scan to send with the request. Moreover, the prompt 506 may include a UI decline button 512 corresponding to an option to decline performing the scan. In response to selecting the UI decline button 512, the mobile application 122 may be configured to cancel the response and provide an indication via the request tool 500 that the request has been cancelled.

FIGS. 6A-6C illustrate front views of respective immune pass 120 formats displayed on a mobile device 124 via the mobile application 122, in accordance with embodiments of the present disclosure. As set forth above, the immunity restricted space 140 may require the individual to provide their corresponding immune pass 120 before allowing the individual to access the immunity restricted space 140. As set forth above, the individual may provide the immune pass 120 to the requestor 152 (e.g., scanner, reader, receiver, etc. shown in FIG. 1) of the immunity restricted space 140 via the application (e.g., mobile application 122 or web application 118) of the immune pass system 100. In particular, the individual may provide the immune pass 120 via the output feature 600 of the application 130 configured to output (e.g., display, transmit, etc.) the immune pass 120 with the device 124 (e.g., mobile phone) running the application 130. The output feature 600 may be initiated via the third UI button 138 associated with the output feature 600 from the main menu 132 of the application 130. In response to selecting the third UI button 138, the output feature 600 is configured to output the immune pass 120.

Referring to FIG. 6A, the illustrated embodiment includes the output feature 600 displaying an image 602 of the immune pass 120 on the screen 604 of the mobile phone 126 running the mobile application 122 of the immune pass system 100. As illustrated, the immune pass 120 includes an indication 606 of the immune pass status corresponding to the individual. As set forth above, the immune pass 120 may include the immunity rating of the individual. In some embodiments, the immune pass 120 may include a pass/fail status corresponding to the immunity levels of the individual, or any other suitable indication of the immune pass status of the individual. Further, the immune pass 120 may include vaccination data for the individual.

Moreover, in the illustrated embodiment, the immune pass 120 includes an indication 608 of personal identifiable information corresponding to the individual. As illustrated, the personal identifiable information includes photo identification, name, age, height, and eye color of the individual. The personal identifiable information may be provided as part of the immune pass 120 to help the requestor 152 at an immunity restricted space 140 verify that the immune pass status of the immune pass 120 corresponds to the individual presenting the immune pass 120 to the requestor 152. The immune pass 120 may be configured to provide any suitable personal identifiable information or contact information, in conjunction with the immune pass status.

Referring to FIG. 6B, the illustrated embodiment includes the output feature 600 displaying the immune pass 120 having a quick response (QR) code 610 on the screen 604 of the mobile phone 126 running the mobile application 122 of the immune pass system 100. The QR code 610 may include the immune pass status, the immunity rating, personal identifiable information, contact information, or some combination thereof for the individual. Moreover, as set forth above, the immunity restricted space 140 may include a requestor 152 (e.g., scanner), which may be configured to scan the QR code 610. Based on the scan of the QR code 610, the immunity restricted space 140 may be configured to permit entrance of the individual to the immunity restricted space 140.

Referring to FIG. 6C, the illustrated embodiment includes the output feature 600 displaying the immune pass 120 having a keycode 612 on the screen 604 of the mobile phone 126 running the mobile application 122 of the immune pass system 100. The keycode 612 may be configured to provide access to the immunity restricted space 140. For example, the keycode 612 may correspond to a keyless lock having a keypad for entering the keycode 612. Upon arrival to the immunity restricted space 140, the individual may output a request for the immune pass 120 with criteria corresponding to the immunity restricted space 140. The criteria may indicate that that the immunity restricted space 140 requires the keycode 612 for entry. Based at least in part on the criteria, the immune pass system 100 may request the keycode 612 from the immune pass account corresponding to the immunity restricted space 140. In some embodiments, the keycode 612 for the immunity restricted space 140 may be stored in the secure data storage system 102. The immune pass system 100 may be configured to output the keycode 612 in response to the immune pass status or immunity rating, of the individual corresponding to the request, satisfying the criteria for the immunity restricted space 140. Moreover, in some embodiments, the immunity restricted space 140 include the virtual space 154 such that the keycode 612 may function as a password to access a mobile application, web site, web application, etc. corresponding to the virtual space 154.

Accordingly, the present disclosure may provide immune pass systems and methods for managing (e.g., uploading, storing, requesting, transmitting, and providing) immunity data for at least one virus (e.g., SARS-CoV-2, SARS-CoV-1, MERS, H1N1, or HIV) via a global network. The methods and systems set forth above may include any of the various features disclosed herein, including one or more of the following statements.

Statement 1. A method for managing an immune pass may comprise receiving uploaded immunization data for at least one individual, wherein the immunization data comprises data indicating antibody levels for a particular virus; analyzing the immunization data to determine an immune pass status for the at least one individual; generating an immune pass for the at least one individual, wherein the immune pass includes the immune pass status and personal identifiable information for the at least one individual; storing the immune pass; verifying a request to retrieve the immune pass corresponding to the at least one individual; and transmitting the immune pass in response to verification of the request.

Statement 2. The method of statement 1, wherein the immunization data comprises data indicating antibody levels for individuals to the SARS-CoV-2 virus.

Statement 3. The method of statement 1, wherein the immune pass status includes a pass status or a fail status, wherein the pass status indicates that the at least one individual has an antibody level above a set threshold.

Statement 4. The method of statement 1, wherein the immune pass status includes an antibody rating, wherein the antibody rating is determined based at least in part on a rating system for antibody levels.

Statement 5. The method of statement 1, comprising the step of re-analyzing the immunization data to determine an immune pass status for the at least one individual in response to an adjustment of a set threshold or rating system for antibody levels.

Statement 6. The method of statement 1, wherein storing the immune pass comprises storing the immune pass in a secure data storage system, wherein the secure data storage system is accessible via internet.

Statement 7. The method of statement 1, wherein the request to retrieve the immune pass includes verification data, wherein the verification data comprises biometric data of the at least one individual, and wherein the biometric data is obtained via a mobile device configured to send the request to retrieve the immune pass.

Statement 8. The method of statement 1, wherein the immune pass is transmitted to a device that sent the request, via a software application, to retrieve the immune pass.

Statement 9. The method of statement 8, wherein the device includes a mobile phone, and wherein the software application includes a mobile application installed on the mobile phone.

Statement 10. A method for providing an immune pass may comprise transmitting a request to retrieve an immune pass, via a mobile application, corresponding to an individual associated with an account logged into the mobile application, wherein the immune pass includes an immune pass status determined based at least in part on antibody levels of the individual for a particular virus; receiving the immune pass from a secure data storage system; and providing the immune pass to an immune pass requestor for access to an immunity restricted space.

Statement 11. The method of statement 10, wherein the mobile application is configured to run on a mobile device, and wherein providing the immune pass includes displaying the immune pass via a display of the mobile device.

Statement 12. The method of statement 10, wherein the immune pass includes a quick response (QR) code comprising the immune pass status and/or personal identifiable information for the individual.

Statement 13. The method of statement 10, wherein the immune pass includes an indication of the immune pass status and personal identifiable information corresponding to the individual.

Statement 14. The method of statement 13, wherein the personal identifiable information includes photo identification of the individual.

Statement 15. The method of statement 10, wherein the immune pass includes a keycode, and wherein the keycode is configured to provide access to the restricted space.

Statement 16. The method of statement 10, wherein the immunity restricted space includes a physical space having a corresponding geofenced area, and wherein the mobile application is configured to provide the immune pass in response to entering the geofenced area.

Statement 17. The method of statement 10, wherein the immunity restricted space includes a virtual space.

Statement 18. A mobile application system may comprise a user interface configured to display the mobile application on a mobile device; an immunity test upload tool configured to upload immunization data for the individual, wherein the immunization data comprises data indicating antibody levels, B-cell levels, and/or T-cell levels, for a particular virus; a request tool configured to output a request, wherein the request to retrieve an immune pass corresponding to the individual associated with the account logged into the mobile application, wherein the immune pass includes an immune pass status determined based at least in part on the immunization data of the individual for a particular virus; and an output feature configured to output the immune pass.

Statement 19. The system of statement 18, wherein the output feature is configured to output the immune pass via wireless communication.

Statement 20. The system of statement 18, wherein the immunization data comprises data indicating a vaccination status for the individual to the particular virus.

Statement 21. The system of statement 18, wherein the mobile application includes a map feature configured to overlay the geofenced area over a map, and wherein the map feature includes tool tips providing information regarding immune pass requirements, wherein the immune pass requirements include time since last request, level of immunity, acceptable immune pass, or some combination thereof, for the geofenced location.

Statement 22. The system of statement 18, wherein the request tool is configured to perform a biometric scan to enable sending of the request.

Although specific embodiments have been described above, these embodiments are not intended to limit the scope of the present disclosure, even where only a single embodiment is described with respect to a particular feature. Examples of features provided in the disclosure are intended to be illustrative rather than restrictive unless stated otherwise. The above description is intended to cover such alternatives, modifications, and equivalents as would be apparent to a person skilled in the art having the benefit of this disclosure.

The scope of the present disclosure includes any feature or combination of features disclosed herein (either explicitly or implicitly), or any generalization thereof, whether or not it mitigates any or all of the problems addressed herein. Various advantages of the present disclosure have been described herein, but embodiments may provide some, all, or none of such advantages, or may provide other advantages.

What is claimed is:

1. A method for managing an immune pass, comprising:
receiving uploaded immunization data for at least one individual, wherein the immunization data comprises data indicating antibody levels for a particular virus;
analyzing the immunization data to determine an immune pass status for the at least one individual;
generating an immune pass for the at least one individual, wherein the immune pass includes the immune pass status and personal identifiable information for the at least one individual;
storing the immune pass;
verifying a request to retrieve the immune pass corresponding to the at least one individual; and
transmitting the immune pass in response to verification of the request.

2. The method of claim 1, wherein the immunization data comprises data indicating antibody levels for individuals to the SARS-CoV-2 virus.

3. The method of claim 1, wherein the immune pass status includes a pass status or a fail status, wherein the pass status indicates that the at least one individual has an antibody level above a set threshold.

4. The method of claim 1, wherein the immune pass status includes an antibody rating, wherein the antibody rating is determined based at least in part on a rating system for antibody levels.

5. The method of claim 1, comprising the step of re-analyzing the immunization data to determine an immune pass status for the at least one individual in response to an adjustment of a set threshold or rating system for antibody levels.

6. The method of claim 1, wherein storing the immune pass comprises storing the immune pass in a secure data storage system, wherein the secure data storage system is accessible via internet.

7. The method of claim 1, wherein the request to retrieve the immune pass includes verification data, wherein the verification data comprises biometric data of the at least one individual, and wherein the biometric data is obtained via a mobile device configured to send the request to retrieve the immune pass.

8. The method of claim 1, wherein the immune pass is transmitted to a device that sent the request, via a software application, to retrieve the immune pass.

9. The method of claim 8, wherein the device includes a mobile phone, and wherein the software application includes a mobile application installed on the mobile phone.

10. A method for providing an immune pass, comprising:
transmitting a request to retrieve an immune pass, via a mobile application, corresponding to an individual associated with an account logged into the mobile application, wherein the immune pass includes an immune pass status determined based at least in part on antibody levels of the individual for a particular virus;
receiving the immune pass from a secure data storage system; and
providing the immune pass to an immune pass requestor for access to an immunity restricted space.

11. The method of claim 10, wherein the mobile application is configured to run on a mobile device, and wherein providing the immune pass includes displaying the immune pass via a display of the mobile device.

12. The method of claim 10, wherein the immune pass includes a quick response (QR) code comprising the immune pass status and/or personal identifiable information for the individual.

13. The method of claim 10, wherein the immune pass includes an indication of the immune pass status and personal identifiable information corresponding to the individual.

14. The method of claim 13, wherein the personal identifiable information includes photo identification of the individual.

15. The method of claim 10, wherein the immune pass includes a keycode, and wherein the keycode is configured to provide access to the restricted space.

16. The method of claim 10, wherein the immunity restricted space includes a physical space having a corresponding geofenced area, and wherein the mobile application is configured to provide the immune pass in response to entering the geofenced area.

17. The method of claim 10, wherein the immunity restricted space includes a virtual space.

18. A mobile application system, comprising:
a user interface configured to display the mobile application on a mobile device;
an immunity test upload tool configured to upload immunization data for the individual, wherein the immunization data comprises data indicating antibody levels, B-cell levels, and/or T-cell levels, for a particular virus;
a request tool configured to output a request, wherein the request to retrieve an immune pass corresponding to the individual associated with the account logged into the mobile application, wherein the immune pass includes an immune pass status determined based at least in part on the immunization data of the individual for a particular virus; and
an output feature configured to output the immune pass.

19. The system of claim 18, wherein the output feature is configured to output the immune pass via wireless communication.

20. The system of claim 18, wherein the immunization data comprises data indicating a vaccination status for the individual to the particular virus.

21. The system of claim 18, wherein the mobile application includes a map feature configured to overlay the geofenced area over a map, and wherein the map feature includes tool tips providing information regarding immune pass requirements, wherein the immune pass requirements include time since last request, level of immunity, acceptable immune pass, or some combination thereof, for the geofenced location.

22. The system of claim 18, wherein the request tool is configured to perform a biometric scan to enable sending of the request.
